(19) United States
(12) Patent Application Publication
(10) Pub. No.: US 2026/0053435 A1
(43) Pub. Date: Feb. 26, 2026

(54) Intelligent Pain Management Patch System (IPMPS)

(71) Applicant: Kamal Yousef Ghazal, Riyadh (SA)

(72) Inventor: Kamal Yousef Ghazal, Riyadh (SA)

(21) Appl. No.: 18/823,733

(22) Filed: Sep. 4, 2024

Related U.S. Application Data
(60) Provisional application No. 63/685,273, filed on Aug. 21, 2024.

(51) Int. Cl. A61B 5/00; A61B 5/0205; G16H 40/67; G16H 50/30

(52) U.S. Cl. CPC A61B 5/4839; A61B 5/0205; A61B 5/4824; A61B 5/7264; G16H 40/67; G16H 50/30; A61B 5/6832; A61B 2560/0214

(57) ABSTRACT

The Intelligent Pain Management Patch System (IPMPS) is a wearable medical device designed to deliver personalized and continuous pain relief for palliative care patients. The system integrates advanced sensor technology, a smart drug delivery system, and AI-driven pain assessment. The patch continuously monitors patient physiological parameters, assesses pain levels in real-time using AI, and administers precise doses of analgesic medication. The system includes a user interface for healthcare providers and patients, facilitating remote monitoring and adjustments. The IPMPS offers a non-invasive, personalized approach to managing chronic pain, reducing the need for oral medications and minimizing side effects.

### PROVISIONAL PATENT APPLICATION FILED

This is to confirm that on Aug. 21, 2024, a provisional patent application for the Intelligent Pain Management Patch System (IPMPS) was filed. The provisional patent application number is 63/685,273.

### FIELD OF THE INVENTION

The present invention relates to the field of medical devices, specifically to an intelligent, wearable pain management system designed to provide personalized and continuous pain relief for patients in palliative care. The system integrates advanced sensor technology, smart drug delivery mechanisms, and AI-driven pain assessment algorithms.

### BACKGROUND OF THE INVENTION

Chronic pain is a prevalent issue among palliative care patients, significantly affecting their quality of life. Traditional pain management methods often rely on oral medications, which can lead to inconsistent relief, undesirable side effects, and challenges in dosage regulation. The need for a more effective, personalized, and consistent pain management system is critical.

The Intelligent Pain Management Patch System (IPMPS) aims to address these challenges by offering a wearable device that continuously monitors the patient's condition, assesses pain levels using AI, and administers precise doses of analgesic medication in response to real-time needs.

This system represents a significant advancement in pain management technology.

### SUMMARY OF THE INVENTION

The Intelligent Pain Management Patch System (IPMPS) is an innovative, wearable medical device designed to provide continuous and personalized pain relief. The system comprises the following components:
Wearable Patch: The patch is constructed from high-grade biocompatible materials, specifically medical-grade silicone and hypoallergenic adhesives. These materials are chosen for their durability, flexibility, and skin-friendly properties, ensuring patient comfort and safety during prolonged use. The biocompatibility of these materials has been validated through ISO 10993 testing, ensuring compliance with international standards for medical devices.
Sensor Technology: The patch includes multiple sensors that monitor physiological parameters (e.g., skin temperature, heart rate, sweat composition) and patient activity levels. These sensors provide real-time data on the patient's condition.
Smart Drug Delivery: The patch contains micro-reservoirs of analgesic medication. An AI-driven system controls the release of the medication, ensuring precise dosage based on the patient's current pain level and physiological data.
AI-Driven Pain Assessment: The AI algorithm was developed and trained using extensive datasets, including patient pain records, physiological patterns such as heart rate variability, skin temperature fluctuations, and cortisol levels in sweat. The algorithm underwent rigorous validation through cross-referencing with clinical pain assessment scales and patient-reported outcomes. It continuously processes real-time sensor data, identifying pain trends and predicting the patient's pain level with high accuracy. The data sources include anonymized clinical trials and real-world patient data, ensuring that the algorithm's predictions are robust and adaptable to various patient conditions.
User Interface: The system includes a mobile app that allows healthcare providers and patients to monitor the device's status, adjust settings, and review pain management history.
Power Supply: The patch is equipped with a lightweight rechargeable lithium-ion battery designed to support continuous operation for up to 72 hours on a single charge, depending on the intensity of usage. The battery recharges within 2 hours using the included wireless charging pad, ensuring minimal downtime. This extended battery life is particularly beneficial for palliative care patients who require uninterrupted pain management without frequent recharges.

The IPMPS system is designed to improve pain management by offering a user-friendly, non-invasive solution that provides consistent, personalized care, reducing the need for oral medications and minimizing the risk of side effects.

### DETAILED DESCRIPTION OF THE INVENTION

### Wearable Patch Design:

The patch is constructed from biocompatible materials to ensure patient comfort and safety during prolonged use. It features a flexible, skin-adherent layer that conforms to the body's contours.

The embedded sensors are strategically positioned within the patch to optimize the monitoring of relevant physiological parameters.

### Sensor Technology:

1. Temperature Sensors: Monitor the skin's temperature to detect inflammation or other pain-related symptoms.
2. Heart Rate Sensors: Track the patient's heart rate, providing insight into stress or pain-induced physiological changes.
3. Sweat Sensors: Analyze sweat composition for markers of pain or distress, such as cortisol levels.
4. Motion Sensors: Detect patient movement, activity levels, and rest periods to correlate with pain episodes.

### Smart Drug Delivery System:

The patch contains micro-reservoirs pre-filled with analgesic medication. Each reservoir is connected to a micro-pump that controls drug release.

The AI system calculates the optimal dosage based on real-time data, ensuring precise delivery of medication directly through the skin.

### AI-Driven Pain Assessment:

The AI algorithm is trained on vast datasets of pain-related physiological patterns. It processes real-time data from the sensors, identifying pain trends and predicting the patient's pain level.

The AI continuously adapts its predictions and drug delivery parameters, offering dynamic pain management that evolves with the patient's condition.

### User Interface:

The mobile app provides a dashboard displaying the patient's real-time physiological data, pain levels, and medication history.

Healthcare providers can remotely adjust the device settings, set medication limits, and review the patient's pain management progress.

Patients can use the app to report subjective pain levels, providing additional input to refine the AI's assessments.

### Power Supply:

The patch is equipped with a lightweight, rechargeable lithium-ion battery, ensuring several days of continuous operation.

A wireless charging option is included to facilitate convenient recharging without removing the patch.

### Detailed Description of the Flowcharts

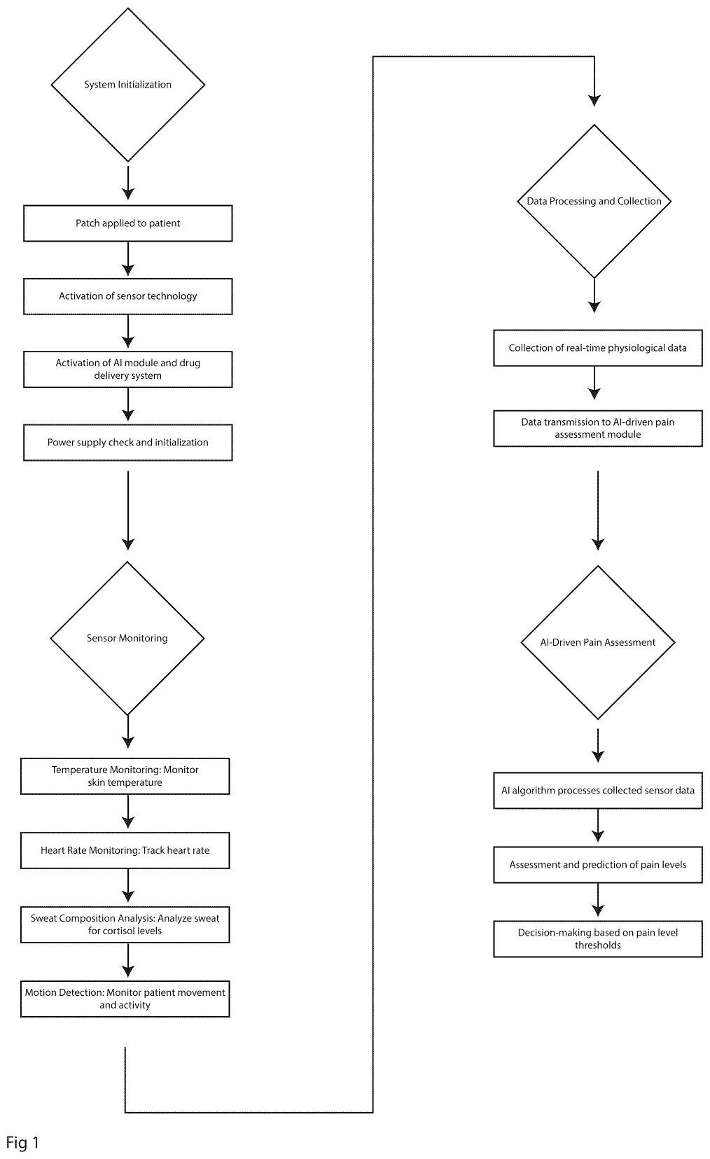

The Intelligent Pain Management Patch System (IPMPS) involves a series of processes as illustrated in the flowcharts provided in FIGS. 1 through 4. These figures detail the system's operation, from initialization to monitoring and smart drug delivery. Below is a detailed explanation of each figure:

### FIG. 1: System Initialization and Sensor Monitoring

System Initialization:
   The process begins when the patch is applied to the patient.
   The sensor technology is activated, followed by the activation of the AI module and drug delivery system.
   A power supply check is conducted to ensure the system is functioning correctly.
Sensor Monitoring:
   Temperature Monitoring: The system monitors skin temperature to detect signs of inflammation or other pain-related symptoms.
   Heart Rate Monitoring: Continuous tracking of the heart rate helps identify stress or pain-induced changes in the patient's physiological state.
   Sweat Composition Analysis: The system analyzes the sweat for cortisol levels, providing data on the patient's stress levels.
   Motion Detection: The system monitors patient movement and activity levels, correlating them with pain episodes.

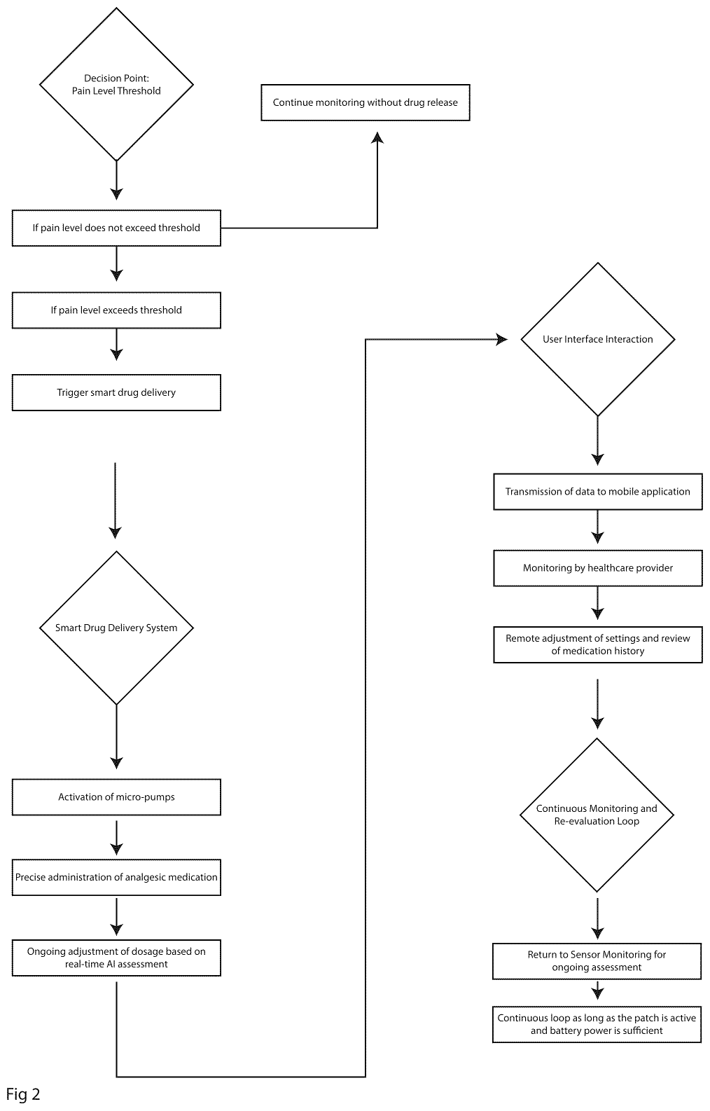

FIG. 2: AI-Driven Pain Assessment and Decision Making
AI-Driven Pain Assessment:
   The AI algorithm processes the sensor data to assess and predict pain levels in real-time.
   Based on the pain level thresholds set within the system, decisions are made regarding drug delivery.
Decision Point—Pain Level Threshold:
   If the pain level exceeds the threshold: The smart drug delivery system is triggered, leading to the activation of micro-pumps and precise administration of analgesic medication.
   If the pain level does not exceed the threshold: The system continues to monitor the patient without releasing any medication.

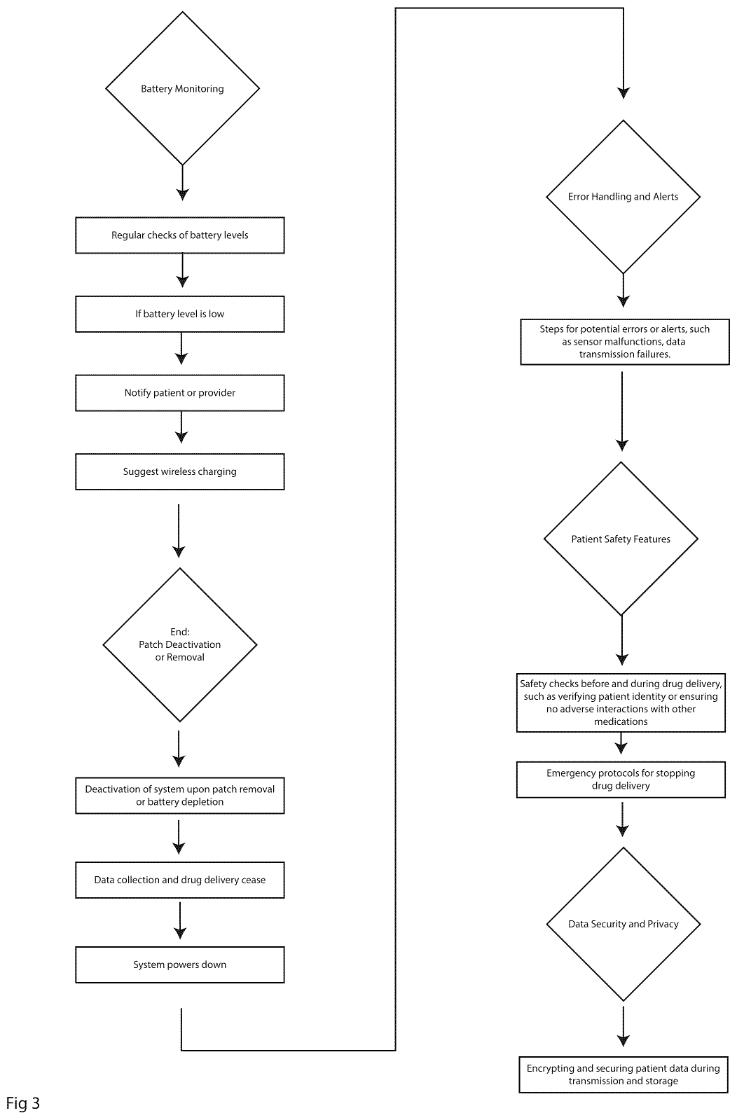

FIG. 3: Smart Drug Delivery and Battery Monitoring
Smart Drug Delivery System:
   The micro-pumps are activated to administer the exact dosage of medication as determined by the AI.
   The dosage is adjusted continually based on real-time assessments, ensuring effective pain management.
Battery Monitoring:
   Regular checks of the battery levels are conducted.
   If the battery level is low, the patient or healthcare provider is notified, and wireless charging is suggested.

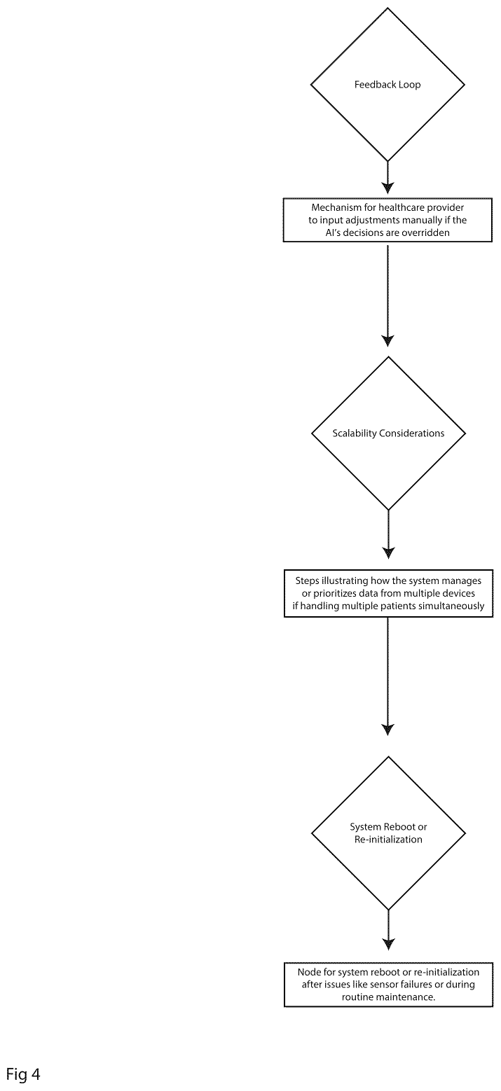

FIG. 4: Continuous Monitoring, Error Handling, and System Re-initialization
Continuous Monitoring and Re-evaluation Loop:
   The system continues to monitor the patient's physiological parameters in a loop, ensuring ongoing assessment and drug delivery as long as the patch is active and the battery power is sufficient.
Error Handling and Alerts:
   The system includes protocols for handling potential errors, such as sensor malfunctions or data transmission failures, to ensure patient safety.
Patient Safety Features:
   Safety checks are performed before and during drug delivery, including verifying patient identity and checking for adverse interactions with other medications.
   Emergency protocols are in place to stop drug delivery if necessary.
Data Security and Privacy:
   The system ensures that all patient data is encrypted and secured during transmission and storage, protecting patient privacy.
System Reboot or Re-initialization:
   If needed, the system can reboot or re-initialize following issues like sensor failures or during routine maintenance.
End of Operation:
   The patch system deactivates upon removal or battery depletion, ceasing all data collection and drug delivery.

1: An intelligent pain management patch system (IPMPS) comprising a wearable patch embedded with sensors, a smart drug delivery system, and an AI-driven pain assessment module, wherein the system monitors patient physiological parameters and provides personalized, real-time pain relief through controlled drug release.

2: The system of claim 1, wherein the wearable patch includes temperature sensors, heart rate sensors, sweat sensors, and motion sensors for monitoring patient condition.

3: The system of claim 1, wherein the smart drug delivery system includes micro-reservoirs of analgesic medication controlled by micro-pumps, with release parameters determined by an AI algorithm.

4: The system of claim 1, wherein the AI-driven pain assessment module processes sensor data to continuously assess and predict the patient's pain level, adjusting medication delivery in real-time.

5: The system of claim 1, further comprising a mobile application that interfaces with the patch, allowing healthcare providers and patients to monitor the system's operation and adjust settings.

6: The system of claim 1, wherein the wearable patch is powered by a rechargeable lithium-ion battery and includes a wireless charging capability.
